(19) United States
(12) Patent Application Publication
(10) Pub. No.: US 2020/0360125 A1
(43) Pub. Date: Nov. 19, 2020

(54) MODULAR VENOUS STENT FOR ILIO-CAVAL RECONSTRUCTION

(71) Applicant: ALEXANDRE C. SAMAK, M.D. INC., Saint-Lambert (CA)

(72) Inventor: Alexandre CENGARLE-SAMAK, Saint-Lambert, CA (US)

(21) Appl. No.: 15/931,834

(22) Filed: May 14, 2020

Related U.S. Application Data
(60) Provisional application No. 62/847,558, filed on May 14, 2019.

(51) Int. Cl. A61F 2/06; A61F 2/954

(52) U.S. Cl. CPC A61F 2/06; A61F 2250/0096; A61F 2002/067; A61F 2/954

(57) ABSTRACT

A stent and a method of deploying thereof, the stent, comprising a main stent configured for deployment in a first passageway; and a limb stent configured for deployment in the main stent and in a second passageway connected to the first passageway; the main stent comprising a constraining septum dividing a lumen of the main stent into compartments, a first one of said compartments being connected to the second passageway.

### CROSS REFERENCE TO RELATED APPLICATIONS

This application claims benefit of U.S. provisional application Serial No. 62/847,558, filed on May 14, 2019. All documents above are incorporated herein in their entirety by reference.

### FIELD OF THE INVENTION

The present invention relates to stents. More specifically, the present invention is concerned with a modular venous stent and a method for ilio-caval reconstruction.

### BACKGROUND OF THE INVENTION

Stents are typically metal or plastic tubes inserted into the lumen of an anatomic vessel or duct to keep the passageway open. There is a wide variety of stents used for different purposes, such as expandable coronary, vascular and biliary stents for example.

Stenting is the placement of a stent within such a passageway. Endovascular ilio-caval reconstruction is an effective treatment for ilio-caval thrombosis or thrombosis of the inferior vena cava and iliac veins. Venous stents are used for the endovascular reconstruction of the distal inferior vena cava and the iliac vein.

Currently used stents are found to be liable to compromise venous flow in the contralateral iliac vein. Allowing the endovascular reconstruction of the distal inferior vena cava and the iliac vein while maintaining integrity of the venous flow in the contralateral iliac, is still a challenge and current reconstruction methods require unsheathing of iliac stents well into the inferior vena cava, at the risk of contralateral iliac vein obstruction or occlusion.

There is still a need in the art for a stent and a method for vessel reconstruction.

### SUMMARY OF THE INVENTION

More specifically, in accordance with the present disclosure, there is provided a stent, comprising a main stent configured for deployment in a first passageway; and a limb stent configured for deployment in the main stent and in a second passageway connected to the first passageway; wherein the main stent comprises a constraining septum, the constraining septum dividing a lumen of the main stent into compartments, a first one of the compartments being connected to the second passageway.

There is further provided a self-expanding non-covered stent comprising a first part comprising a constraining septum and a reversed tapered second part having a first diameter proximally for deployment within the first part and a second diameter distally.

There is further provided, in a configuration comprising a first passageway splitting into at least a second and a third passageways, a method for deploying a stent in the first and second passageways, the method comprising providing a stent comprising a main stent configured for deployment in the first passageway and comprising a constraining septum, the constraining septum dividing a lumen of the main stent into two compartments, and a limb stent comprising a cephalad and a caudal stents; accessing the second passageway; introducing the main stent and the limb stent through the second passageway into the first passageway; and deploying the main stent and the limb stent; whereby the cephalad stent is deployed in the first passageway and the caudal stent is deployed in the second passageway.

There is further provided, in a configuration comprising a first passageway dividing into at least a second and a third passageways, a method for deploying a stent in the first and third passageways, the method comprising: providing a stent comprising a main stent configured for deployment in the first passageway and comprising a constraining septum, the constraining septum dividing a lumen of the main stent into two compartments, and a limb stent comprising a cephalad and a caudal stents; accessing the second passageway; introducing the main stent and the limb stent through the second passageway into the first passageway; accessing the third passageway; selectively catheterizing the main stent; and deploying the main stent and the limb stent; whereby the cephalad stent is deployed in the first passageway and the caudal stent is deployed in the third passageway.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 is a schematical front view of a device according to an embodiment of an aspect of the present disclosure;

FIG. 2 is a front view of a main stent of a device according to an embodiment of an aspect of the present disclosure;

FIG. 3 is a top view of the main stent of FIG. 2;

FIG. 4 is a lateral view of the limb stent of a device according to an embodiment of an aspect of the present disclosure;

FIG. 5A shows a top cross-section of the limb stent of FIG. 4;

FIG. 5B shows a bottom cross-section of the limb stent of FIG. 4;

FIG. 6A is a lateral view of a constraining septum of a main stent of the device according to an embodiment of an aspect of the present disclosure;

FIG. 6B is a lateral view of a constraining septum of a main stent of the device according to an embodiment of an aspect of the present disclosure; and

FIG. 7 is a front plan view of the device in a deployed position thereof according to an embodiment of an aspect of the present disclosure.

### DESCRIPTION OF ILLUSTRATIVE EMBODIMENTS

The present invention is illustrated in further details by the following non-limiting examples.

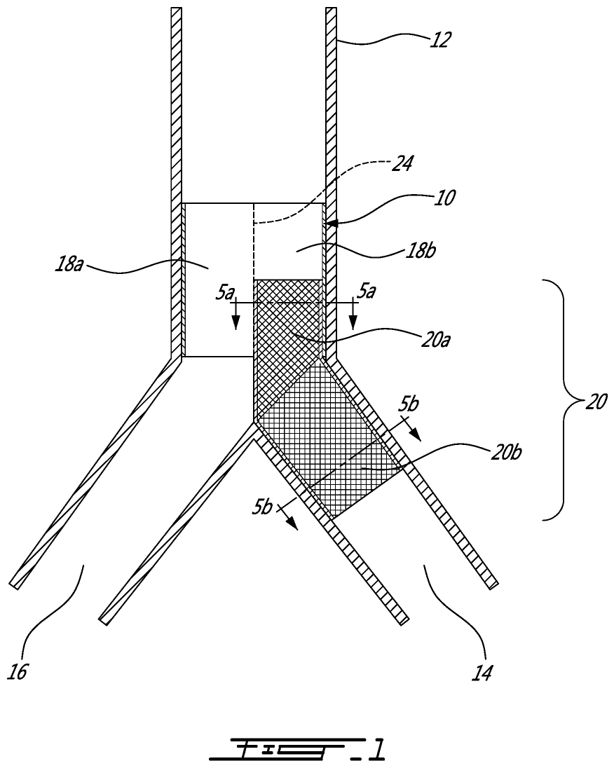

The device generally comprises a main stent 10 and a limb stent 20. In the following example, the limb stent 20 will be described as an ipsilateral iliac stent 20. FIG. 1 shows a device according to an embodiment of an aspect of the present disclosure, positioned in the region where the inferior vena cava 12 separates into the contralateral right iliac vein 16 and the left iliac vein 14.

The main stent 10 is a self-expanding tubular body. It is a non-covered stent. It is made in a material selected for crush resistance and high radial force so as to withstand the external pressure caused by the overlying iliac artery 12 when in position. The material of the main stent 10 may be stainless steel or nitinol for example. The size of the cells of the walls of the main stent 10 is selected to minimize risks of reendothelialization and minimize the impact of the of the main stent 10 on the fluid dynamics within the venous system.

The limb stent 20 comprises a cephalad stent 20a configured for unsheathing in the main stent 10 and a caudal stent 20b configured for unsheathing in the main stent 10 as will be described hereinbelow.

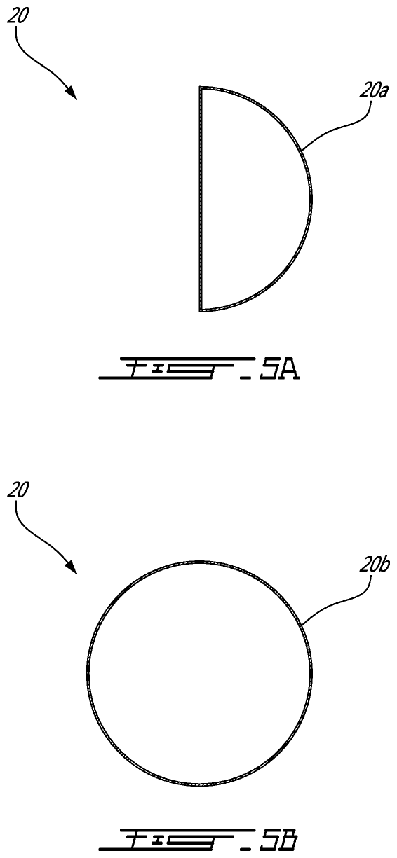

As shown in FIG. 5A, the cephalad stent 20a is a half-tubular body having a semi-circular cross section matching the semi-circular cross section of a compartment of the main stent 10. As shown in FIG. 5B, the caudal stent 20b is a tubular body having a circular section corresponding to the circular section of the native iliac vein 14 (see circular bottom cross section in FIG. 5B) for unsheathing into the native iliac vein 14 from the cephalad stent 20a. When fully deployed, with the cephalad stent 20a deployed in the main stent 10 and the caudal stent 20b deployed in the native iliac vein 14, the diameter of the cross section of the limb stent within the native iliac vein 14 is larger than the diameter of the cross section the end of the limb stent within the main stent 10, and the deployed body of the limb stent 20 is an inverted tapered body.

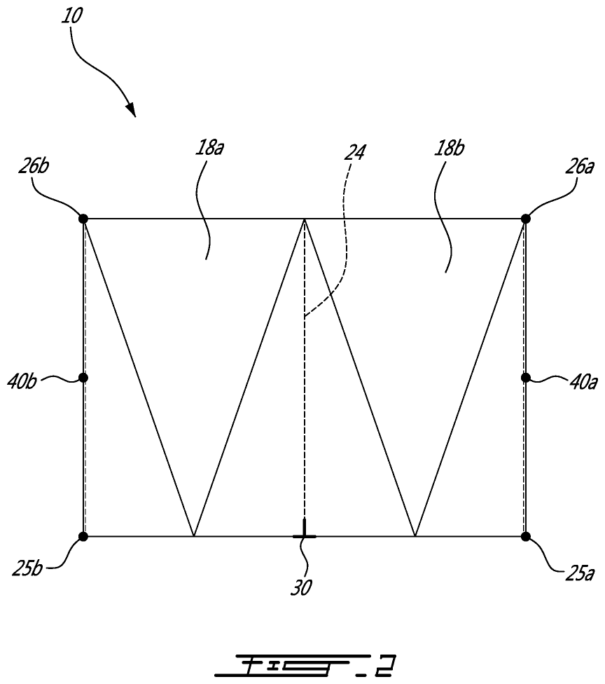

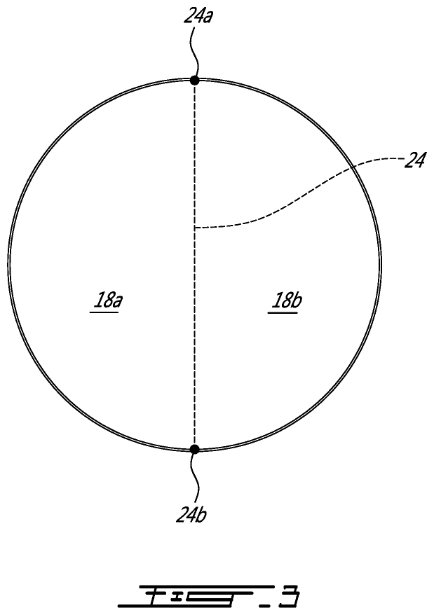

As shown in FIGS. 1 to 3, the circular lumen of the main stent 10 is divided into two semi-circular compartments 18a, 18b by a non-covered midline septum 24 (see FIGS. 2 and 3). The septum 24 may be welded or sutured to the main stent 10, as best seen in FIG. 3 (see connections 24a and 24b in FIG. 3).

Once the main stent 10 is deployed within the inferior vena cava 12, the midline septum 24 prevents the ipsilateral iliac stent 20 from tilting and/or protruding over the ostium of the right iliac vein 16 by constraining the cephalad stent 20a in place within the left compartment 18b of the main stent 10

The septum 24 is selected with cells of a size selected to minimize the risk of reendothelialization and minimize the impact of the of the main stent 10 on the natural fluid dynamics within the venous system (see FIG. 2).

The septum 24 allows the operator to selectively stent the right iliac vein 16, for example in the case of a complete bilateral ilio caval occlusion, or the left iliac vein 14, for example, in the case of a May-Thurner syndrome.

FIG. 2 shows markers on the main stent 10, provided to monitor the precise the position and orientation of the device upon unsheathing of the stents thereof, thereby guiding the positioning of the device.

The markers may be in a metal, for example gold, denser than the material of the main stent 10 and the limb stent 20 so that they are X-ray visible.

In FIG. 2, markers 26a, 26b mark the cephalad edge of the main stent 10. An inverted T marker 30 indicates the midline position of the septum 24, to control the midline unsheathing of the main stent 10 within the inferior vena cava 12. Top limb markers 40a, 40b mark the level of the target unsheathing of the cephalad stent 20a of the limb stent 20. Illio-caval junction markers 25a, 25b mark the bottom edge of the main stent 10.

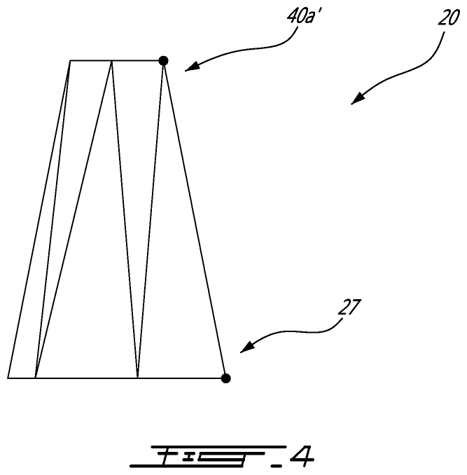

FIG. 4 shows a side view of the ipsilateral limb stent 20 when fully deployed, a top marker 40a′, marks the top edge of the cephalad stent 20a. Distal iliac markers 27 mark the bottom edge of the caudal stent 20b.

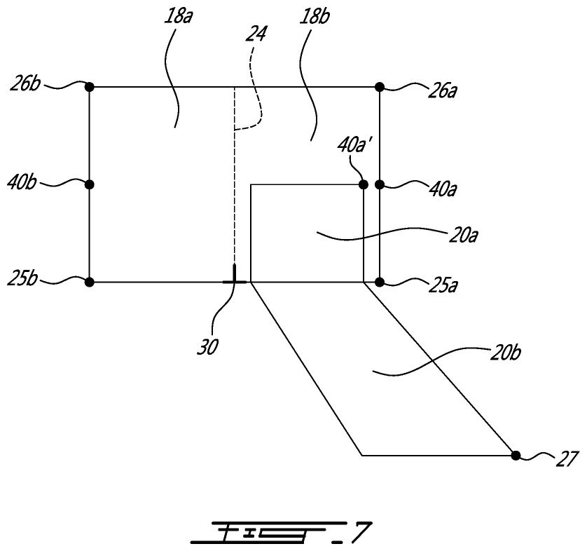

FIG. 7 shows the markers in a deployed form of the device.

Unless the goal is to reconstruct the ilio-caval junction upwards, the ilio-caval junction is deployed for alignment with the natural ilio-caval junction for conformance with the native anatomy of the body. In such case, the main body stent 10 is positioned so as to align the ilio-caval marker 25a with the native lateral ilio-caval junction. The main body is deployed by unsheathing. The ipsilateral limb stent 20t is then advanced co-axially, over a wire. The top marker 40a′ on the ipsilateral limb stent 20 is then aligned with the top limb marker 40a on the main stent 10 and the ipsilateral limb stent 20 is deployed in the left iliac vein 14 so that the distal marker 27 reaches the left iliac vein 14.

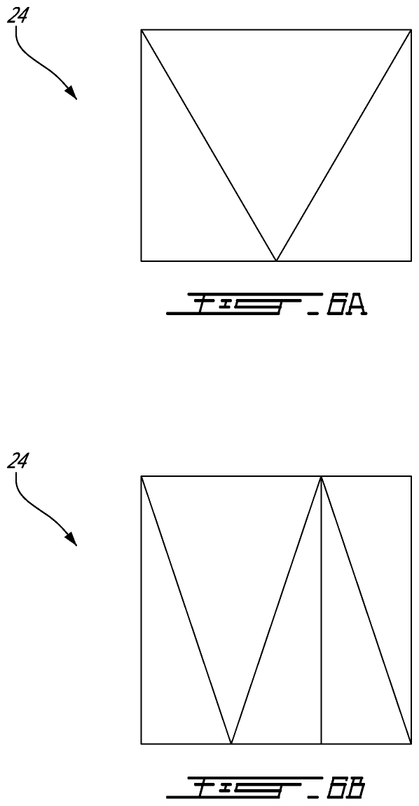

FIG. 6 show examples of cells formed by struts of the walls of the main stent 10 and of the septum 24, or the walls of the cephalad stent 20a of the limb stent 20. By adjusting the density of the struts, wider or narrower cell may be selected, so as not to interfere with blood circulation, which may lead to formation of a membrane between the struts. The cells of the walls of the caudal stent 20b of the limb stent 20 may be of a smaller size in order to increase mechanical resistance.

As people in the art will appreciate, the present stent device may be deployed in the distal inferior vena cava. The central constraining septum of the main stent prevents the iliac stent from straddling the ostium of the right iliac vein 16 by directing the stent cephalad 20a. The right iliac vein 16 may thus also be stented for example in the case of a complete bilateral ilio cavel occlusion, or the left iliac vein 14 may thus be selectively stented in the case of treating May Thurner syndrome.

There is thus presented a device comprising a self-expanding non-covered main stent comprising a central constraining septum, and a limb stent, for unsheathing in both the inferior vena cava (main stent) and in the native iliac vein (ipsilateral iliac stent). The limb stent is a reversed tapered stent to be deployed within the main stent with a first diameter proximally and having a second, larger diameter distally (see FIG. 4).

In a method for stenting the left iliac vein 14 according to the present disclosure, percutaneous access at the left iliac vein 14 is obtained. Venography if performed, delineating the anatomy. The stent device as described hereinabove is positioned by introducing the main stent and ipsilateral limb through the left iliac vein 14 up into the inferior vena cava 12, until alignment of the Ilio-caval junction marker 25a with the natural ilio-caval junction. The main stent 10 and the limb stent 20 are then deployed by unsheathing. Venography is performed to confirm adequate unsheathing and landing of the stent device. Post-unsheathing balloon dilatation using a selectively sized balloon catheter may be performed.

In a method for stenting the right contralateral iliac vein 16 according to the present disclosure, the main stent 10 and left iliac stent 20 are deployed though a percutaneous access at the left iliac vein 14 as described above for stenting the iliac vein 14. Percutaneous access is then obtained at the right contralateral iliac vein 16. The main stent is then selectively catheterized. A guidewire with a wide J shape may be used to prevent crossing of the main stent 10 through the septum cells, which would result in the unsheathing of the limb stent, intended for unsheathing in the right compartment 18a, into the left compartment 18b of the main stent 10. The limb stent 20 is deployed by unsheathing, once the top limb marker 40a′ is laterally aligned with the marker 40b of the main stent 10. Venography is performed to confirm adequate unsheathing and landing of the stent. Post-unsheathing balloon dilatation may be performed with an appropriate sized balloon catheter.

The present stent device comprising a constraining septum is a self-expanding non-covered stent allowing unsheathing both an inferior vena cava stent and an ipsilateral iliac stent for example. The present self-expanding non-covered ipsilateral iliac stent has a reversed tapered geometry, with a first diameter proximally and a second, larger diameter distally. The cephalad part of the limb stent has a semi-circular cross-section, whereas the caudal port of the limb stent has a circular cross-section.

The scope of the claims should not be limited by the embodiments set forth in the examples, but should be given the broadest interpretation consistent with the description as a whole.

1. A stent, comprising:
a main stent configured for deployment in a first passageway; and
a limb stent configured for deployment in said main stent and in a second passageway connected to the first passageway;
wherein the main stent comprises a constraining septum, said constraining septum dividing a lumen of the main stent into compartments, a first one of said compartments being connected to said second passageway.

2. The stent of claim 1, wherein said main stent is a self-expanding tubular body.

3. The stent of claim 1, wherein said main stent is a non-covered stent.

4. The stent of claim 1, wherein said limb stent comprises a cephalad stent configured for deployment in the main stent and a caudal stent configured for deployment in the second passageway.

5. The stent of claim 1, wherein said limb stent comprises a cephalad stent and a caudal stent, said cephalad stent being a body having a cross section matching a cross section of said of the main stent; and said caudal stent is a body having a cross section matching a cross section of said second passageway; wherein when said limb stent is deployed, said cephalad stent is deployed in the main stent and the caudal stent is deployed in the second passageway, and wherein when said limb stent is deployed, said limb stent has a diameter of a cross section of en end thereof within the second passageway different than a diameter of a cross section of an end thereof within the main stent.

6. The stent of claim 1, wherein said limb stent comprises a cephalad stent and a caudal stent, said cephalad stent has a cross section matching a cross section of said first compartment of the main stent;
and said caudal stent has a cross section matching a cross section of said second passageway; and wherein when said limb stent is deployed, said limb stent is an inverted tapered body.

7. The stent of claim 1, wherein said limb stent comprises a cephalad stent and a caudal stent, said cephalad stent having a cross section matching a cross section of said first compartment of the main stent; and said caudal stent having a cross section matching a cross section of said second passageway; wherein when said limb stent is deployed, said cephalad stent is deployed in the main stent and the caudal stent is deployed in the second passageway; and when said limb stent is deployed, said limb stent is an inverted tapered body is an inverted tapered body, said inverted tapered body having a first end cross section matching a cross section of the first compartment of the main stent and a second end cross section matching a cross section of the second passageway.

8. The stent of claim 1, wherein said main stent is a self-expanding non-covered tubular body; said constraining septum divides said lumen of said main stent into two compartments of a semi-circular cross-section; said limb stent comprises a cephalad stent and a caudal stent, said cephalad stent is a half-tubular body having a semi-circular cross section matching a semi-circular cross section of the first compartment of the main stent; and said caudal stent is a tubular body having a circular section matching a cross section of the second passageway.

9. The stent of claim 1, wherein said main stent comprises first markers and said limb stent comprises second markers, said first and second markers indicating relative positioning of said main and limb stents when said stent is deployed.

10. The stent of claim 1, wherein said limb stent comprises a cephalad stent configured for deployment in the main stent and a caudal stent configured for deployment in the second passageway; said main stent comprises at least one of: markers marking a cephalad edge of the main stent in said first passageway, markers marking a position of the septum within the main stent, markers marking a deployed position of the cephalad stent within the main stent and markers marking a bottom edge of the main stent within the first passageway; said limb stent comprising at least one of: markers marking a top edge of the cephalad stent in said main stent and markers marking a bottom edge of the caudal stent within the second passageway.

11. A self-expanding non-covered stent comprising a first part comprising a constraining septum and a reversed tapered second part having a first diameter proximally for deployment within the first part and a second diameter distally.

12. In a configuration comprising a first passageway splitting into at least a second and a third passageways, a method for deploying a stent in the first and second passageways, the method comprising :
providing a stent comprising a main stent configured for deployment in the first passageway and comprising a constraining septum, the constraining septum dividing a lumen of the main stent into two compartments, and a limb stent comprising a cephalad and a caudal stents;
accessing the second passageway;
introducing the main stent and the limb stent through the second passageway into the first passageway; and
deploying the main stent and the limb stent;
whereby the cephalad stent is deployed in the first passageway and the caudal stent is deployed in the second passageway.

13. The method of claim 12, wherein the main stent is a self-expanding non-covered tubular body and the constraining septum divides the main stent into two compartments of a semi-circular cross-section; the cephalad stent is a half-tubular body having a semi-circular cross section matching a semi-circular cross section of a first one of the compartments of the main stent; and the caudal stent is a tubular body having a circular section matching a cross section of the second passageway.

14. In a configuration comprising a first passageway dividing into at least a second and a third passageways, a method for deploying a stent in the first and third passageways, the method comprising :
providing a stent comprising a main stent configured for deployment in the first passageway and comprising a constraining septum, the constraining septum dividing a lumen of the main stent into two compartments, and a limb stent comprising a cephalad and a caudal stents,;
accessing the second passageway;
introducing the main stent and the limb stent through the second passageway into the first passageway;
accessing the third passageway;
selectively catheterizing the main stent; and
deploying the main stent and the limb stent;
whereby the cephalad stent is deployed in the first passageway and the caudal stent is deployed in the third passageway.

15. The method of claim 14, wherein the main stent is a self-expanding non-covered tubular body and the constraining septum divides the main stent into two compartments of a semi-circular cross-section;
wherein the cephalad stent is a half-tubular body having a semi-circular cross section matching a semi-circular cross section of a first one of the compartments of the main stent; and the caudal stent is a tubular body having a circular section matching a cross section of the third passageway.
